(19) United States
(12) Patent Application Publication
(10) Pub. No.: US 2019/0046662 A1
(43) Pub. Date: Feb. 14, 2019

(54) Compositions and Methods for Treating Neuropathic Pain

(71) Applicant: Rutgers, The State University of New Jersey, New Brunswick, NJ (US)

(72) Inventor: Gary Aston-Jones, Highland Park, NJ (US)

(73) Assignee: Rutgers, The State University of New Jersey, New Brunswick, NJ (US)

(21) Appl. No.: 16/078,324

(22) PCT Filed: Mar. 24, 2017
(86) PCT No.: PCT/US2017/023987

§ 371 Date: Aug. 21, 2018

Related U.S. Application Data
(60) Provisional application No. 62/312,716, filed on Mar. 24, 2016.

(51) Int. Cl. A61K 48/00; A61K 35/761; A61K 31/551; A61K 31/366; C07K 14/705; A61P 25/02

(52) U.S. Cl. CPC A61K 48/0058; A61K 35/761; A61K 48/0075; A61P 25/02; A61K 31/366; C07K 14/705; A61K 31/551

(57) ABSTRACT

Compositions and methods for treating, inhibiting, and/or preventing pain, particularly neuropathic pain, are provided.

This application claims priority under 35 U.S.C. § 119(e) to U.S. Provisional Patent Application No. 62/312,716, filed Mar. 24, 2016. The foregoing application is incorporated by reference herein.

### FIELD OF THE INVENTION

The present invention relates to the field of pain. Specifically, compositions and methods for inhibiting, treating, and/or preventing pain, particularly neuropathic pain, are disclosed.

### BACKGROUND OF THE INVENTION

Several publications and patent documents are cited throughout the specification in order to describe the state of the art to which this invention pertains. Each of these citations is incorporated herein by reference as though set forth in full.

Neuropathic pain (NP) typically arises as a consequence of a lesion or disease affecting nerve fibers such as peripheral nerve fibers. In painful traumatic trigeminal neuropathies (PTTN), the triggering event is a traumatic lesion of the trigeminal nerve, which can lead to NP.

NP is a common disease that is extremely difficult to manage. A best estimate for the population prevalence of NP is between 6.9-10% (van Hecke et al. (2014) 155:654-662). Following routine root canal treatment (which induces a mild nerve injury), PTTN has been found in ˜3% of patients (Klasser et al. (2011) Quintessence Int., 42:259-69). Additionally, NP is a frequent component of many neurological disorders, affecting 20-40% of patients with primary neurological diseases such as Parkinson's and multiple sclerosis (Salter, M. W. (2014) Brain 137:651-3; Devor, M. (2005) “Response of nerves to injury in relation to neuropathic pain”, in Wall and Melzack's Textbook of Pain, M. Koltzenburg and S. B. McMahon, Eds., Churchill Livingstone: Edinburgh). These diseases result from a wide range of pathophysiologies including traumatic injury to the central nervous system, neurodegeneration and neuroinflammation.

The initiation and establishment of PTTN involves molecular mechanisms leading to increased neuronal activity (Fried et al. (2001) Neuroscientist 7:155-65; Salter, M. W. (2014) Brain 137:651-3). This is clearly seen in ectopic activity originating from the injured site and the trigeminal ganglion (TG) (spontaneous pain) and a decreased threshold to stimuli (induced pain) (Fried et al. (2001) Neuroscientist 7:155-65; Salter, M. W. (2014) Brain 137:651-3). Additional changes alter the phenotype of sensory nerves such that non-pain fibers start to induce pain on stimulation and interact with the sympathetic nervous system to further augment pain (Devor, M. (2005) “Response of nerves to injury in relation to neuropathic pain”, in Wall and Melzack's Textbook of Pain, M. Koltzenburg and S. B. McMahon, Eds., Churchill Livingstone: Edinburgh; Devor et al. (2009) Exp. Brain Res., 196:115-28). Changes in CNS pain modulatory areas lead to decreased pain inhibition and increased facilitation. Ectopic and increased neuronal activities in the peripheral afferent and associated soma (e.g., TG) are considered important pathophysiologic features of PTTN and trigeminal neuralgia (Fried et al. (2001) Neuroscientist 7:155-65; Salter, M. W. (2014) Brain 137:651-3; Rappaport et al. (1994) Pain 56:127-38).

In general, NP is frequently refractory to conventional analgesics and currently there are no effective treatments (O'Connor et al. (2009) Am. J. Med., 122:S22-32). Management of chronic NP requires long-term prescription medications that have significant side effects (McDermott et al. (2006) Eur. J. Pain 10:127-35). Unfortunately even with standard of care protocols, significant relief is obtained in only ˜10-15% of PTTN patients (Haviv et al. (2014) J. Oral Facial Pain Headache 28:52-60). NP and its current treatment therefore lead to impaired quality of life, reduced employment, low productivity and extensive usage of healthcare services (McDermott et al. (2006) Eur. J. Pain 10:127-35; Meyer-Rosberg et al. (2001) Eur. J. Pain 5:379-89).

### SUMMARY OF THE INVENTION

In accordance with the present invention, compositions and methods for inhibiting, treating, and/or preventing pain, particularly neuropathic pain, are provided. In a particular embodiment, the method comprises administering to a subject at least one Designer Receptor Exclusively Activated by Designer Drug (DREADD) encoding nucleic acid and an agonist for the DREADD. In a particular embodiment, the DREADD is Gi coupled. In a particular embodiment, the DREADD is hM4D1 and the agonist is clozapine N-oxide (CNO) or an analog thereof. In a particular embodiment, the DREADD is KORD and the agonist is salvanorin B or analog thereof. The nucleic acid encoding the DREADD may be contained within a vector, particularly a viral vector (e.g., AAV), and may be operably linked to or under the control of a neuron specific promoter (e.g., synapsin promoter or tachykinin 1 promoter). In a particular embodiment, the neuron specific promoter is the synapsin promoter. In a particular embodiment, the neuropathic pain is associated with a painful traumatic trigeminal neuropathy (PTTN). In a particular embodiment, the neuropathic pain is a peripheral neuropathic pain or a neuropathic cancer pain.

Vectors and compositions for performing the methods of the instant invention are also provided.

### BRIEF DESCRIPTIONS OF THE DRAWING

FIG. 1 provides a sequence of the Tac1 promoter with an upstream enhancer. The underlined sequences are restriction sites MluI and KpnI. The sequence in italics is the upstream enhancer sequence.

FIG. 2A provides an image of trigeminal ganglion with a nuclear stain (DAPI) and the fluorescent mCherry marking the expressed Gi receptor. The Gi was located within the cytoplasm of many neuronal cell bodies of many sizes. This demonstrates that this method is effective for causing expression of the DREADD in trigeminal ganglion neurons, a component of the invention. FIG. 2B provides a graph of the mean grooming times (±SEM) in the early (acute) phase and tonic phases following formalin injection. Grooming time is a measure of response to facial pain in rodent models. All animals had DREADD injected into the trigeminal ganglion. Following injection with CNO, total grooming time was reduced relative to the animals injected with vehicle (n=3/group). * p<0.04.

FIG. 3 shows the effects of synapsin-DREADD activation with CNO on von Frey detection threshold. Animals had synapsin-DREADD (hSyn-hM4D; synapsin promoter driven DREADD) injected into the right trigeminal ganglion. Average von Frey detection thresholds (±SEM) were plotted following intraperitoneal (i.p.) administration of CNO or vehicle. BL=baseline von Frey detection threshold prior to hSyn-hM4D injection; D28=detection threshold at 28 days post hSyn-hM4D injection at 30 minutes following i.p. administration of CNO or vehicle. * CNO compared to the vehicle injected animals (P<0.05) (ANOVA).

FIG. 4 shows the effects of synapsin-DREADD activation with CNO on pin-prick response score. Animals had synapsin-DREADD (hSyn-hM4D) injected into the right trigeminal ganglion. Average pin-prick response scores (±SEM) are plotted following i.p. administration of CNO or vehicle. BL=baseline von Frey detection threshold prior to hSyn-hM4D injection; D28=detection threshold at 28 days post hSyn-hM4D injection at 30 minutes following i.p. administration of CNO or vehicle. * CNO compared to the vehicle injected animals (P<0.05) (ANOVA).

FIG. 5 shows the effects of synapsin-DREADD activation with CNO on face grooming activity observed after subcutaneous (s.c.) injection of formalin. The mean number of seconds that rats spent grooming (±SEM) is plotted for the 45 minutes post-formalin injection observation period. All animals had synapsin-DREADD (hSyn-hM4D) injected into the right trigeminal ganglion. CNO or vehicle was delivered intraperitoneally 30 minutes prior to the subcutaneous injection of formalin into the right upper lip (ipsilateral side), just lateral to the nose. * CNO compared to the vehicle injected animals (P<0.05) (ANOVA).

FIG. 6 shows the effects of synapsin-DREADD activation with CNO on pain-like behavior following infraorbital nerve chronic constriction injury (IoN-CCI). The mean von Frey detection thresholds (±SEM) (left) and the mean response scores (±SEM) (right) to the pin-prick stimulation applied to the vibrissal pads were plotted. Animals were exposed to the IoN-CCI unilaterally. Following IoN-CCI, all animals had synapsin-DREADD (hSyn-hM4D) injected into the ipsilateral trigeminal ganglion. At post-operative day 26 (D26) CNO or vehicle was delivered intraperitoneally 30 minutes prior to behavioral testing. * CNO compared to either the vehicle injected animals or post-operative days D21 and D27 (P<0.05) (ANOVA).

FIG. 7 shows the effects of Tac1-DREADD activation with CNO on von Frey detection threshold. Animals had tachykinin 1-DREADD (hTac1-hM4D) injected into the right trigeminal ganglion. Average von Frey detection thresholds (±SEM) were plotted following i.p. administration of CNO or vehicle. BL=baseline von Frey detection threshold prior to hSyn-hM4D injection; D28=detection threshold at 28 days post hSyn-hM4D injection at 30 minutes following i.p. administration of CNO or vehicle.

FIG. 8 shows the effects of Tac1-DREADD activation with CNO on pin-prick response score. Animals had tachykinin 1-DREADD (hTac1-hM4D) injected into the trigeminal ganglion unilaterally. Average pin-prick response scores (±SEM) were plotted following i.p. administration of CNO or vehicle. BL=baseline von Frey detection threshold prior to hSyn-hM4D injection; D28=detection threshold at 28 days post hSyn-hM4D injection at 30 minutes following i.p. administration of CNO or vehicle.

FIG. 9 shows the effects of Tac1-DREADD activation with CNO on face grooming activity observed after s.c. injection of formalin into the right upper lip. The mean number of seconds that rats spent grooming (±SEM) is plotted for the 45 minutes post-formalin injection observation period. Animals had Tac1-DREADD (hTac1-hM4D) injected into the right trigeminal ganglion. CNO or vehicle was delivered intraperitoneally 30 minutes prior to the s.c. injection of formalin into the right upper lip.

FIG. 10 shows the effects of Tac1-DREADD activation with CNO on pain-like behavior following IoN-CCI. The mean von Frey detection thresholds (±SEM) (left) and the mean response scores (±SEM) (right) to the pin-prick stimulation applied to the vibrissal pads were plotted. Animals were exposed to the IoN-CCI unilaterally. Following IoN-CCI, all animals had Tachykinin 1-DREADD (hTac1-hM4D) injected into the ipsilateral trigeminal ganglion. At post-operative day 26 (D26) CNO or vehicle was delivered intraperitoneally 30 minutes prior to behavioral testing. * CNO compared to either the vehicle injected animals or post-operative days D21 and D27 (P<0.05) (ANOVA).

### DETAILED DESCRIPTION OF THE INVENTION

Neuropathic pain (NP) is a common disease that can arise as a direct consequence of a lesion or disease affecting the somatosensory system. It is most often associated with an injury or a disease affecting nerve fibers, particularly peripheral nerve fibers. Prominent features in the pathophysiology of neuropathic pain are ectopic and increased neuronal activity. Currently there are no effective NP treatments available. The management of painful traumatic trigeminal neuropathies (PTTN) relies on pharmacologic agents with severe side effects and very low success rates.

Herein, new therapeutic methods are provided which use a new class of synthetic receptors termed DREADDs (Designer Receptors Exclusively Activated by Designer Drugs). The chemogenetic approach of the instant invention has been used to achieve localized inhibition and suppression of neuronal excitability via expression of a synthetic Gi-coupled, G protein-coupled receptor (GPCR) (e.g., hM4Di). These receptors (e.g., hM4Di) are activated by an otherwise inert ligand (e.g., clozapine N-oxide (CNO)). Notably, the receptor, particularly hM4Di, is essentially insensitive to acetylcholine or other brain chemicals. Thus, the receptor exhibits no effects in the absence of its selective agonist (CNO). The ability to target and transiently suppress neuronal excitability, without permanently altering other neuronal properties, via the systemic delivery of a small molecule inhibitor makes the instant invention an attractive, focused gene therapy approach for neuropathic pain.

As described hereinbelow, a rat model of trigeminal neuropathic pain was used to investigate the use of hM4Di as therapeutic agents to treat neuropathic pain. A viral vector containing the hM4Di gene was microinjected into the trigeminal ganglion (TG). hM4Di expression, transport of hM4Di receptors along TG axons, and effectiveness of stimulating these DREADDs using systemic CNO in producing focal analgesia and alleviating experimental PTTN were studied in the rat model.

The cell bodies of all primary trigeminal afferent nociceptors lie within the TG. Therefore, in addition to having an important role in pathologic pain, the TG is a strategic locus where afferent input can be successfully manipulated. Successful transfection of rat TG by viral vector microinjection has been demonstrated (Vit et al. (2009) Mol. Pain 5:42). An AAV carrying the glutamic acid decarboxylase (GAD) gene was expressed in satellite glial cells of the TG and induced GABA production. This GABA was thought to act on neurons to induce analgesia. However, as in many such systems, the expression was constitutive which has significant consequences associated with uncontrolled expression and uncontrolled alteration of function.

The instant invention provides a more attractive therapeutic method which involves the transfection of a new class of receptors termed DREADDs (Designer Receptors Exclusively Activated by Designer Drugs), particularly hM4Di (Armbruster et al. (2007) Proc. Natl. Acad. Sci., 104:5163-8; Dong et al. (2010) Mol. Biosystems 6:1376-80; Krashes et al. (2011) J. Clin. Invest., 121:1424-1428; Zhu et al. (2014) Neuropsychopharmacology 39:1880-1892; Roth, B.L. (2016) Neuron 89:683-694; each of the foregoing references is incorporated herein by reference for description of DREADDs and hM4Di). In a particular embodiment, the DREADD is an engineered G-protein coupled receptor which is activated by otherwise inert drug-like small molecules. This technique combines chemical and genetic approaches to achieve localized and temporally specified decreases in neuronal excitability by viral expression of the synthetic receptor (e.g., hM4Di) (Katzel et al. (2014) Nat. Commun., 5:3847; Mahler et al. (2014) Nat. Neurosci., 17:577-85; Pei et al. (2008) Physiology 23:313-21; Ferguson et al. (2011) Nat. Neurosci., 14: 22-24; each of the foregoing references is incorporated herein by reference for description of DREADDs and hM4Di). The hM4Di receptor is a modified human muscarinic receptor (muscarinic acetylcholine receptor) that couples normally to Gi signaling cascades and GIRK channels, but is insensitive to acetylcholine, muscarin, or other endogenous compounds (Pei et al. (2008) Physiology 23:313-21). However, this synthetic receptor is strongly activated by the otherwise pharmacologically inert ligand, clozapine N-oxide (CNO). Furthermore, specificity achieved by regional and cell-type specific expression of DREADDs allows for targeted and temporally limited suppression of neuronal excitability.

Thus, the instant invention provides a genetically encoded and highly selective ‘lock-and-key’ approach to controlling aberrant neural function for therapeutic goals. Through activation of these DREADDs via systemic or local CNO, neural hyperactivity and pain sensitivity, as well as adenylyl cyclase and cAMP levels, in TG neurons will be attenuated. In other words, hM4Di receptors can be activated in a dose-dependent manner by their agonist CNO, thereby allowing for flexible modulation of TG function. This ability to modulate TG function allows for producing pain relief while safeguarding normal sensation in clinical application. The hM4Di receptor is strongly inhibitory in most neurons, due to its strong stimulation of GIRK membrane channels. Thus, the hM4Di receptor will dampen hyperactivity of TG neurons following injury and/or inflammation in a CNO dose-dependent fashion.

The instant invention encompasses methods of inhibiting, treating, and/or preventing pain in a subject. In a particular embodiment, the pain is neuropathic pain. In a particular embodiment, the pain is neuropathic peripheral pain. In a particular embodiment, the pain is neuropathic cancer pain. In a particular embodiment, the methods comprise administering a nucleic acid molecule encoding a DREADD and administering an agonist of the DREADD to the subject. In a particular embodiment, the nucleic acid molecule encoding the DREADD is administered prior to the administration of the agonist. The methods of the instant invention may also comprise administering at least one other therapeutic for the treatment of pain (e.g., an analgesic). In a particular embodiment, the DREADD agonist and/or DREADD nucleic acid is delivered as a composition with at least one pharmaceutically acceptable carrier. When the DREADD agonist and DREADD nucleic acid molecule are delivered in separate compositions, the pharmaceutically acceptable carrier for the compositions may be different.

As explained herein, the pain to be treated, inhibited, and/or prevented by the compositions and methods of the instant invention may be a neuralgia or neuropathic pain. In a particular embodiment, the neuropathic pain is painful traumatic trigeminal neuropathy (PTTN). In a particular embodiment, the neuropathic pain is a peripheral neuropathic pain. In a particular embodiment, the neuropathic pain is a component of a disease or disorder, particularly a neurological disease or disorder (e.g., neurodegenerative disease, Parkinson's Disease, multiple sclerosis, traumatic injury, neuroinflammation, dysmyelination, leukodystrophies, Guillan-Barre syndrome, Charcot-Marie Tooth type I disease, type 2 diabetes, etc.) or cancer.

The nucleic acids of the instant invention may be delivered to a cell (e.g., neuron) by any known method. For example, the nucleic acids can be delivered via synthetic delivery systems, liposomes, nanoparticles, or viral vectors. The nucleic acid molecules encoding the DREADD may be contained within an expression vector, particularly a viral vector such as an adenoviral vector. The nucleic acid molecules (or vectors) may be directly delivered to the target neurons (e.g., by microinjection). For example, the nucleic acid molecules or vectors may be administered (e.g., by injection) to the trigeminal ganglion or dorsal root ganglion (e.g., directly or proximately). Examples of viral vectors include, without limitation, lentiviral, retroviral, herpesviral (e.g., replication-defective herpes simplex virus (HSV)), and adenoviral vectors. In a particular embodiment, the vector is an AAV vector. The AAV vector can be of any AAV serotype. For example, the AAV vector can be, without limitation, AAV1, AAV2, AAV3, AAV4, AAV5, AAV6, AAV7, AAV8, AAV9, AAV10, AAV11, AAV12, and hybrids thereof (e.g., a combinatorial hybrid of 2, 3, 4, 5, or more serotypes). AAV vectors can have one or more of the AAV wild-type genes deleted in whole or part (e.g., the rep and/or cap genes). In a particular embodiment, the AAV vector is AAV5.

As explained hereinabove, DREADDs are engineered G-protein coupled receptors which are activated by otherwise inert drug-like small molecules. In a particular embodiment, the DREADD is based on the muscarinic receptor (e.g., human muscarinic receptor). In a particular embodiment, the DREADD is a KORD (kappa opioid receptor-DREADD (e.g., human KOR)) (Marchant et al. (2016) Neuropsychopharmacology 41(2):402-9). For example, the KORD may be a G-protein coupled (e.g., Gi-coupled) kappa-opioid receptor DREADD wherein the inert ligand or agonist is salvinorin B (salB) (see, e.g., Vardy et al. (2015) Neuron 86:936-946; incorporated herein by reference). In a particular embodiment, the DREADD is coupled with Gi. In a particular embodiment, the DREADD is hM4Di (human M4 muscarinic cholinergic Gi-coupled DREADD; described in references cited hereinabove). In a particular embodiment, the DREADD is human muscarinic acetylcholine receptor M4 (e.g., GenBank Accession No. NP_000732, Gene ID: 1132) comprising two point mutations: a substitution at Y113 (e.g., Y113C) and a substitution at A203 (e.g., A203G). WO 2015/136247 (incorporated herein by reference) also provides a nucleic acid sequence encoding hMD4i (see, e.g., SEQ ID NO: 1 of WO 2015/136247) and a plasmid encoding hM4Di is available commercially as plasmid 45548 from Addgene, Cambridge, Mass. (www.addgene.org/45548/).

The DREADD encoding nucleic acid may be operably linked to or under the control of a neuron specific promoter. In a particular embodiment, the DREADD encoding nucleic acid is under the control of a TG neuron specific promoter. In a particular embodiment, the DREADD encoding nucleic acid is under the control of an A-delta neuron promoter. In a particular embodiment, the DREADD encoding nucleic acid is under the control of a Substance P (SP) neuron promoter. Expression of the receptor only in Substance P neurons provides an additional degree of specificity for attenuation of pain versus non-pain tactile function. SP is nearly exclusively found in neurons that convey pain information. Therefore, DREADD expression in SP neurons (e.g., when driven by the Tac1 promoter) will provide a means for specifically inhibiting primarily neurons that are involved in pain processing. This provides a selective and clinically useful therapeutic.

In a particular embodiment, the neuron specific promoter is the toll-like receptor 5 (TLRS) promoter. TLRS is expressed in large diameter A-fiber neurons in the dorsal root ganglion (Xu et al. (Nat. Med. (2015) 21(11):1326-31; see also GenBank Gene ID: 7100).

In a particular embodiment, the neuron specific promoter is a synapsin promoter (e.g., Kugler et al. (2003) Gene Ther., 10:337-47; GenBank Gene ID: 6853). In a particular embodiment, the neuron specific promoter is a synapsin I promoter. As shown herein, the synapsin promoter (e.g., human synapsin promoter) drives production of DREADD (e.g., hM4Di) in a large percentage of TG neurons.

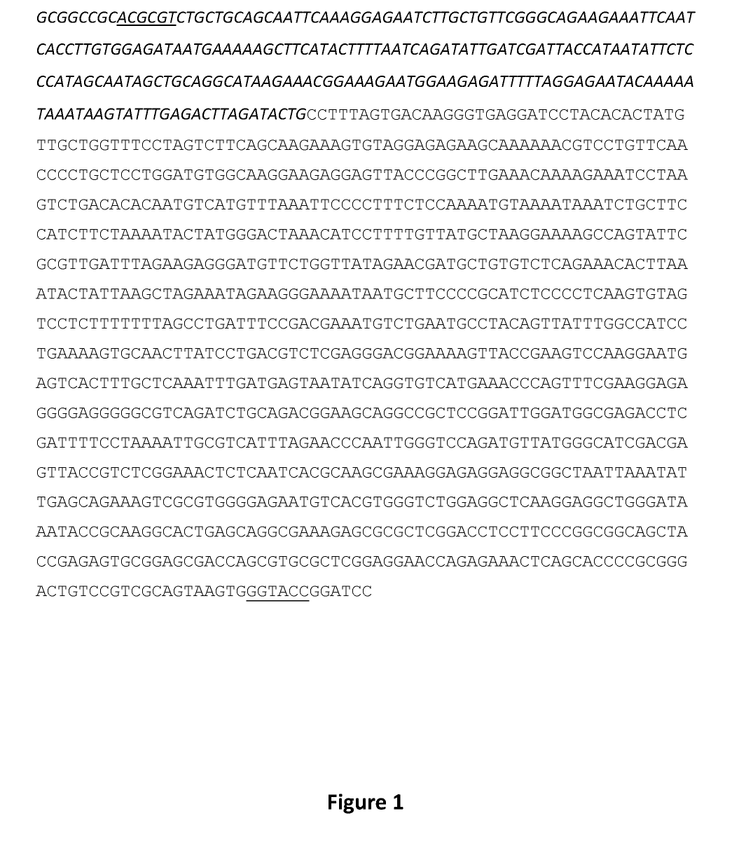

In a particular embodiment, the neuron specific promoter is the tachykinin precursor 1 (Tac1; NCBI Gene ID: 6863) promoter. Tac1 promoter-driven vectors will produce expression in fewer neurons, but these will be tachykinin (e.g., SP) positive. More specifically, the use of the Tac1-promoter vector allows for the targeting of peptidergic fibers (largely C and some A-delta). Specifically targeting peptidergic fibers significantly attenuates pain (Benoliel et al. (1999) Pain 79:243-53). Further, DREADD (e.g., hM4Di) receptors will be transported along efferent axons to peripheral TG fibers. These will be restricted to SP-positive fibers in cases expressing the Tac1-driven gene. The promoter sequence for the Tac1 promoter (e.g., the Tac1 promoter can be considered to lie between −865 to +92 of the Tac1 gene) has been described (Shanley et al. (2010) Neurosignals 18:173-85) and this promoter has been used in viral vectors to express other genes in neurons (Hikida et al. (2010) Neuron 66:896-907; Delzor et al. (2012) Hum. Gene Ther. Methods 23:242-54). In a particular embodiment, the Tac1 promoter has at least 80%, 85%, 90%, 92%, 95%, 98%, 99%, or 100% identity with the sequence provided in FIG. 1 (SEQ ID NO: 1), particularly the sequence within the identified restriction sites. The sequence provided in FIG. 1 provides the Tac1 promoter with an upstream enhancer.

The agonist of DREADD preferentially binds and activates the administered DREADD receptor over other receptors (e.g., a selective agonist). It is desirable to use a DREADD agonist which is inert or has little or no biological effects other than stimulating the DREADD. For example, the agonist may be a ligand of the DREADD. In a particular embodiment, the agonist is CNO, DREADD compound/agonist 21 (Tocris (Bristol, UK); 11-(1-piperazinyl)-5H-dibenzo[b,e][1,4]diazepine); Chen et al. (2015) ACS Chem. Neurosci., 6(3):476-84), or perlapine (Tocris; 6-(4-methyl-1-piperazinyl)-11H-dibenz[b,e]azepine; Chen et al. (2015) ACS Chem. Neurosci., 6(3):476-84) (Roth, Neuron (2016) 89(4): 683-694). In a particular embodiment, the agonist is clozapine N-oxide (CNO) or analog thereof. In a particular embodiment, the agonist is salvinorin B (salB) or analog thereof. The DREADD agonist may be delivered peripherally and/or systemically to the subject (e.g., orally, topically (e.g., to the skin), etc.) or directly to the area of pain (e.g., injection, topically (e.g., to the skin), etc.).

The compositions of the present invention can be administered by any suitable route, for example, by injection (e.g., for local, direct, or systemic administration), oral, pulmonary, topical (e.g., skin cream or lotion), nasal or other modes of administration. The composition may be administered by any suitable means, including oral, parenteral, intramuscular, intravascular, intravenous, intraarterial, intraperitoneal, subcutaneous, topical, inhalatory, transdermal, intrapulmonary, intraareterial, intrarectal, intramuscular, and intranasal administration. In a particular embodiment, the nucleic acid composition is administered by injection (e.g., microinjection directly to neurons). In a particular embodiment, the composition comprising the agonist is administered systemically or is administered topically, orally, or to the eye.

In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences and Remington: The Science and Practice of Pharmacy). The pharmaceutical composition of the present invention can be prepared, for example, in liquid form, or can be in dried powder form (e.g., lyophilized for later reconstitution).

The therapeutic agents described herein (e.g., DREADD nucleic acid molecules and/or DREADD agonists) will generally be administered to a patient as a pharmaceutical preparation. The term “patient” as used herein refers to human or animal subjects. The compositions of the instant invention may be employed therapeutically or prophylactically, under the guidance of a physician.

The compositions comprising the agent of the instant invention may be conveniently formulated for administration with any pharmaceutically acceptable carrier(s). The concentration of agent in the chosen medium may be varied and the medium may be chosen based on the desired route of administration of the pharmaceutical preparation. Except insofar as any conventional media or agent is incompatible with the agent to be administered, its use in the pharmaceutical preparation is contemplated.

The dose and dosage regimen of the agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient (e.g., DREADD agonist and/or nucleic acid molecule encoding DREADD) calculated to produce the desired effect in association with the selected pharmaceutical carrier. Procedures for determining the appropriate dosage unit are well known to those skilled in the art. For example, dosage units may be proportionately increased or decreased based on the weight of the patient. Appropriate concentrations for alleviation or prevention of a particular condition (e.g., pain) may be determined by dosage concentration curve calculations, as known in the art.

The pharmaceutical preparation comprising the therapeutic agent (e.g., DREADD agonist and/or nucleic acid molecule encoding DREADD) may be administered at appropriate intervals, for example, at least twice a day or more until the pathological symptoms are reduced or alleviated, after which the dosage may be reduced to a maintenance level. In a particular embodiment, the nucleic acid molecule encoding the DREADD may be administered more than once in order to ensure sufficient expression of DREADD throughout the target tissue. In a particular embodiment, the DREADD agonist may be administered as needed to reduce or alleviate pathological symptoms (e.g., pain). The appropriate interval in a particular case would normally depend on the condition of the patient (e.g., amount of pain). With regard to prevention or reduction of pain, the compositions of the instant invention may be administered in doses at appropriate intervals based on pain levels.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a therapeutically or prophylactically effective amount.

### Definitions

The following definitions are provided to facilitate an understanding of the present invention:

The singular forms “a,” “an,” and “the” include plural referents unless the context clearly dictates otherwise.

“Pain” includes, without limitation, nociceptive pain, neuropathic pain, psychogenic pain, pain due to functional disorders, and the like. The term “neuropathic pain” refers to pain due to irritation or damage or disorders of the peripheral or central nerve system and results from lesions or diseases affecting the somatosensory system. Neuropathic pain includes, without limitation, peripheral neuropathic pain, central neuropathic pain, and mixed neuropathic pain.

As used herein, the term “cancer pain” or “neuropathic cancer pain” refers to pain, particularly peripheral neuropathic pain, arising or resulting from cancer. The pain can be caused by the destruction of tissue in any region associated with cancer or its metastases; pressure or compression caused by cancer (e.g., tumors) on bones, organs or nerves; inflammation caused by cancer; and/or chemicals secreted by cancerous cells and/or tissues. In a particular embodiment, the term “cancer pain” also encompasses neuropathic pain caused indirectly by cancer treatments such as radiation therapy and chemotherapy.

As used herein, the term “agonist” refers to an agent (e.g., ligand, protein, polypeptide, peptide, lipid, antibody, antibody fragment, large molecule, or small molecule) that binds to a receptor and has an intrinsic effect such as inducing a receptor-mediated response. For example, the agonist may stimulate, increase, activate, facilitate, enhance, or up regulate the activity of the receptor. In a particular embodiment, the agonist is a ligand.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “vector” refers to a carrier nucleic acid molecule (e.g., RNA or DNA) into which a nucleic acid sequence can be inserted for introduction into a host cell where it will be replicated. The vector may be an integrating vector or a non-integrating vector. Examples of vectors include, without limitation, plasmids, phagemids, cosmids, and viral vectors. An “expression vector” is a specialized vector that contains a gene or nucleic acid sequence with the necessary regulatory regions needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., pain (e.g., neuropathic pain)) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to modulate pain in a subject.

As used herein, the term “subject” refers to an animal, particularly a mammal, particularly a human.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain without a loss of consciousness). Analgesics include opioid analgesics (e.g., codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine) and non-opiate analgesics (e.g., NSAIDs such as salicylates (e.g., aspirin, methyl salicylate, and diflunisal); arylalkanoic acids (e.g., indomethacin, sulindac, diclofenac, and tolmetin); N-arylanthranilic acids (e.g., fenamic acids, mefenamic acid, and mecflofenamate); oxicams (e.g., piroxicam and meloxicam); coxibs (e.g., celecoxib, rofecoxib, valdecoxib, parecoxib, and etoricoxib); sulphonanilides (e.g., nimesulide); naphthylalkanones (e.g., nabumetone); anthranilic acids (e.g., pyrazolidinediones and phenylbutazone); proprionic acids (e.g., fenoprofen, flurbiprofen, ibuprofen, ketoprofen, naproxen, and oxaprozin); pyranocarboxylic acids (e.g., etodolac); pyrrolizine carboxylic acids (e.g., ketorolac); and carboxylic acids.

The following examples provide illustrative methods of practicing the instant invention and are not intended to limit the scope of the invention in any way.

### EXAMPLE 1

Rats received stereotaxically-guided microinjections of AAV5-hSyn-hM4Di(Gi)-mCherry into TG unilaterally as described (Mahler et al. (2014) Nat. Neurosci., 17:577-85; Karai et al. (2004) J. Clin, Invest., 113:1344-52; Vazey et al. (2014) Proc. Natl. Acad. Sci., 111:3859-64; Fortress et al. (2015) J. Neurosci., 35:1343-53; Delzor et al. (2012) Hum. Gene Ther. Methods 23:242-54). The injection cannula (Karai et al. (2004) J. Clin. Invest., 113:1344-52; Benoliel et al. (2003) “Viral transfection of trigeminal ganglia in rats,” in International Association for Dental Research: Jerusalem, Israel) were inserted until the base of the skull was reached (˜10-11 mm). Then 1 μl of AAV5-hSyn-hM4Di-mCherry was infused over a period of 10 minutes and allowed to diffuse for an additional 10 minutes. Rats recovered >4 weeks with no observable adverse effects.

For the immunohistochemistry (IHC) study, rats were deeply anesthetized and then transcardially perfused with saline and 4% paraformaldehyde (Vazey, et al. (2014) J. Clin. Invest., 124:2858-60; Mogil et al. (2005) Pain 117:1-5). The TG was harvested. Tissue sections through the TG were cut at 10 μm-thickness (Leica cryostat) and thaw-mounted onto slides. To visualize DREADD expression, slides were incubated in rabbit anti-mCherry antibody (1:500, Abcam) overnight, followed by donkey anti-rabbit antibody conjugated to Alexa Fluor® 594 (1:200, Invitrogen) for 2 hours. DAPI staining was used to mark nuclei.

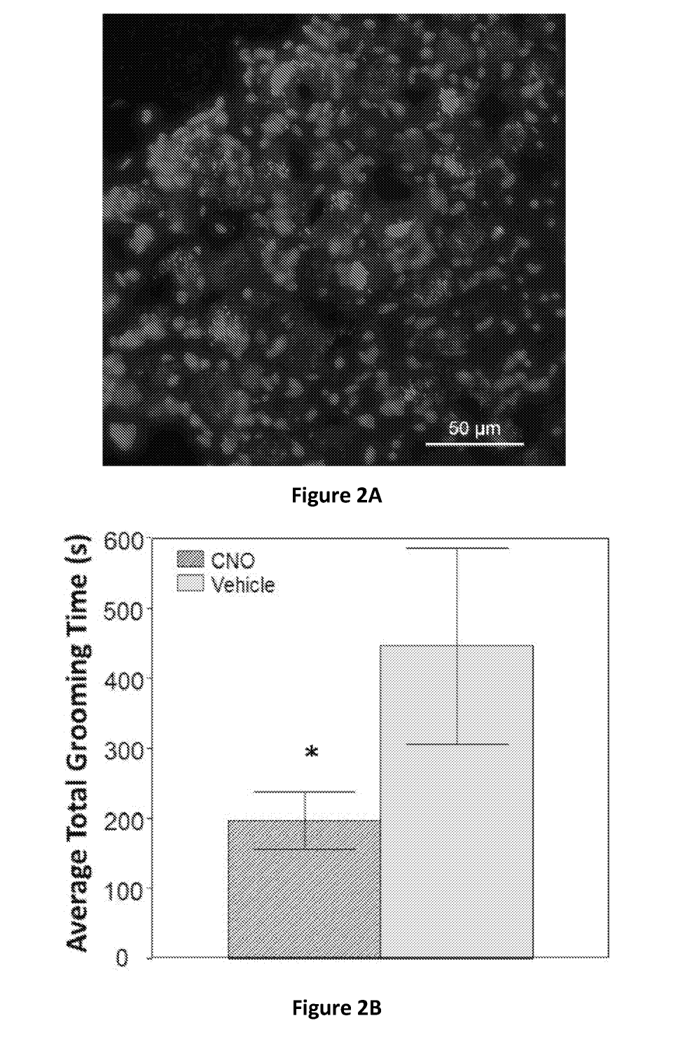

Injections of an AAVS encoding the hM4Di receptor gene under the control of a synapsin promoter produced robust hM4Di expression in TG neurons as seen by the fused mCherry tag (FIG. 2A). The analysis also indicated that hM4Di receptors were transported out of the TG, along TG neural fibers. This indicates that stimulation of peripheral hM4Di receptors would also be effective in treating NP. Notably, the use of the tachykinin promoter resulted in expression within Substance P neuronal subpopulations.

The orofacial formalin model is well established for inducing behavioral and molecular changes indicating pain. The formalin model mimics the clinical situation of an acute insult that wanes rapidly. Models of trigeminal pain and their behavioral and molecular assessments have been described (Benoliel et al. (2001) Pain 91:111-21; Benoliel et al. (2001) Neurosci. Lett., 297:151-4; Benoliel et al. (2002) Pain 97:203-12; Benoliel et al. (2002) Pain 99:567-78). There is increased and intense directed facial grooming following injection of formalin into the rat vibrissal pad, and the duration of grooming is thought to represent the level of pain (Clavelou et al. (1995) Pain 62:295-301; Raboisson et al. (1995) J. Neurophysiol., 73:496-505; Sukriti et al. (2004) Methods Find Exp. Clin. Pharmacol., 26:253-6). At day 37 after virus injection (to allow for viral transfection and DREADD expression), acute pain was induced by subcutaneous injection of 50 μL of 1.5% formalin into the rat's vibrissal pad. Formalin activates Aδ and C nociceptors as well as trigeminal and spinal nociceptive neurons. Formalin-induced unilateral pain is biphasic, with an early short-lasting (3-5 minutes) phase, followed after a quiescent period by a second, prolonged (20-40 minutes) tonic phase. The orofacial formalin test is a valid and reliable way of producing and quantifying nociception in the trigeminal region of the rat (Raboisson et al. (2004) Neurosci. Biobehay. Rev., 28:219-26; Le Bars et al. (2001) Pharmacol. Rev., 53:597-652; Vos et al. (1998) Pain 76:173-8). The concentration of formalin used allows for the detection of both hypo- and hyperalgesic effects. The time that the animal spends rubbing the injected area with fore- or hind paws was recorded. Thirty minutes prior to injecting formalin the animals received CNO (10 mg/ml, ip) or vehicle treatment.

CNO stimulation of hM4Di receptors expressed in TG significantly reduced the total time spent grooming after formalin (FIG. 2B) and produced trends to reduce grooming in both early (<9 minutes) and late phases (9-45 minutes). Thus, the data indicates a robust analgesic effect of activated DREADDs in the formalin model.

### EXAMPLE 2

The infraorbital chronic constriction injury (CCI) model is well established for inducing behavioral and molecular changes indicating pain. The model mimics the clinical situation of persistent neuropathic pain that continues for a number of days or weeks. This model is ideally suited to assess the effects of DREADD activation on pain processes. Based on the robust analgesic effect of activated DREADDs in the formalin model (see above), a significant effect is also predicted in the CCI model, which will allow for the assessment of treatment effects over time with chronic CNO administration (e.g., in drinking water; Cassataro et al. (2014) Neuropsychopharmacology 39:283-90; Jain et al. (2013) J. Clin. Invest., 123:1750-62). As seen in Example 3, the activation of DREADDs with CNO administration resulted in reduction of neuropathic pain in the CCI model.

For the infraorbital nerve chronic constriction injury (ION-CCI), ˜0.5 cm of the infraorbital nerve will be freed and two chromic gut ligatures will be loosely tied around it (2 mm apart) prior to closure of the incision (all performed under ketamine/xylazine anesthesia). As a control, other rats will undergo sham surgery where the infraorbital nerve is exposed but left untouched. This model results in persistent pain beginning at about 2 weeks postoperatively and lasting at least 3 weeks (Benoliel et al. (2001) Neurosci. Lett., 297:151-4; Benoliel et al. (2002) Pain 97:203-12), allowing for the assessment of the effect of vehicle vs CNO/DREADD-mediated inhibition of TG neurons on this behavior at multiple time points. Fos expression and cAMP levels may also be measured in the TG to correlate these molecular endpoints to behavioral effects of DREADD activation. The rats will also undergo repeated stimulation with a 2-g von Frey hair applied to the hairy skin of the vibrissae on the operated side to induce Fos (Vos et al. (1995) J. Comp. Neurol., 357:362-75). Activation of hM4Di receptors will reduce Fos in the TG following von Frey stimulation. As seen in Example 3, the activation of DREADDs with CNO administration resulted in reduction of neuropathic pain in this model.

### EXAMPLE 3

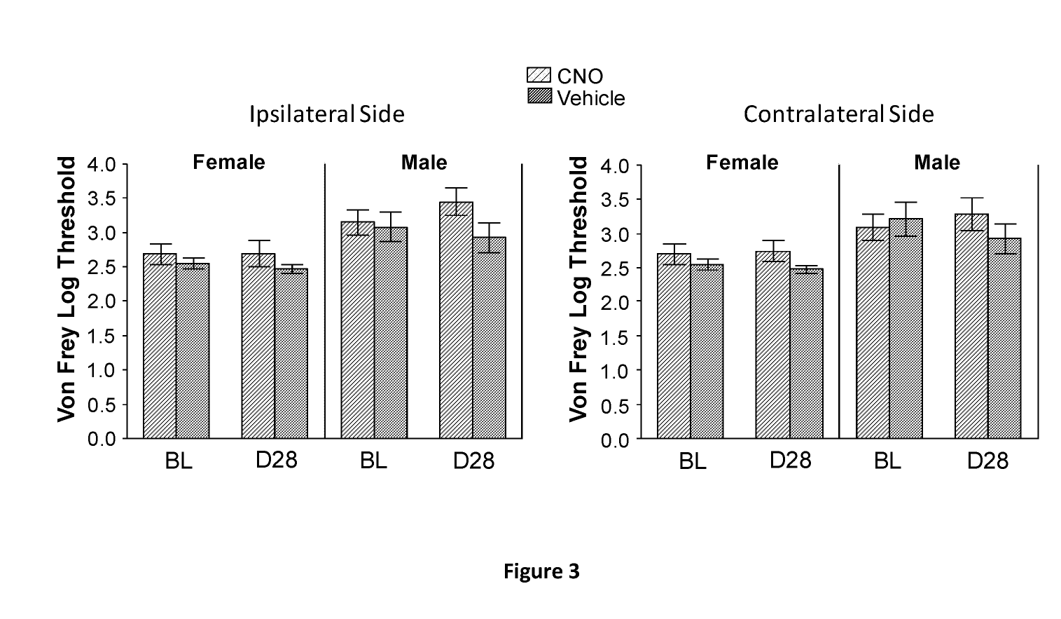

The effects of synapsin-DREADD activation with CNO on von Frey detection threshold were studied (FIG. 3). The von Frey assay uses von Frey fibers, which are small pieces of nylon rod of varying diameters, to test a rodent's sensitivity to a mechanical stimulus. The von Frey assay is generally considered a mechanical nociceptive threshold test. All animals (males, n=7 per group; females, n=7 per group) had synapsin-DREADD (hSyn-hM4D; synapsin promoter driven DREADD) injected into the right trigeminal ganglion. Average von Frey detection thresholds (±SEM) were plotted following intraperitoneal (i.p.) administration of CNO or vehicle. In both sexes, activation of hSyn-hM4D with CNO had no significant effect on von Frey detection thresholds in either ipsilateral or contralateral vibrissal pad compared to the von Frey thresholds in the ipsilateral and contralateral sides of vehicle treated animals, respectively (females: ipsilateral, ANOVA: treatment F=1.2, P=0.3; contralateral, ANOVA: treatment F=2.8, P=0.1; males: ipsilateral, ANOVA: treatment F=3.3, P=0.1; contralateral, ANOVA: treatment F=1.2, P=0.3). This indicates that the synapsin promoter driven vector does not strongly suppress normal (non-chronic compression injury, CCI, animals) sensory detection thresholds.

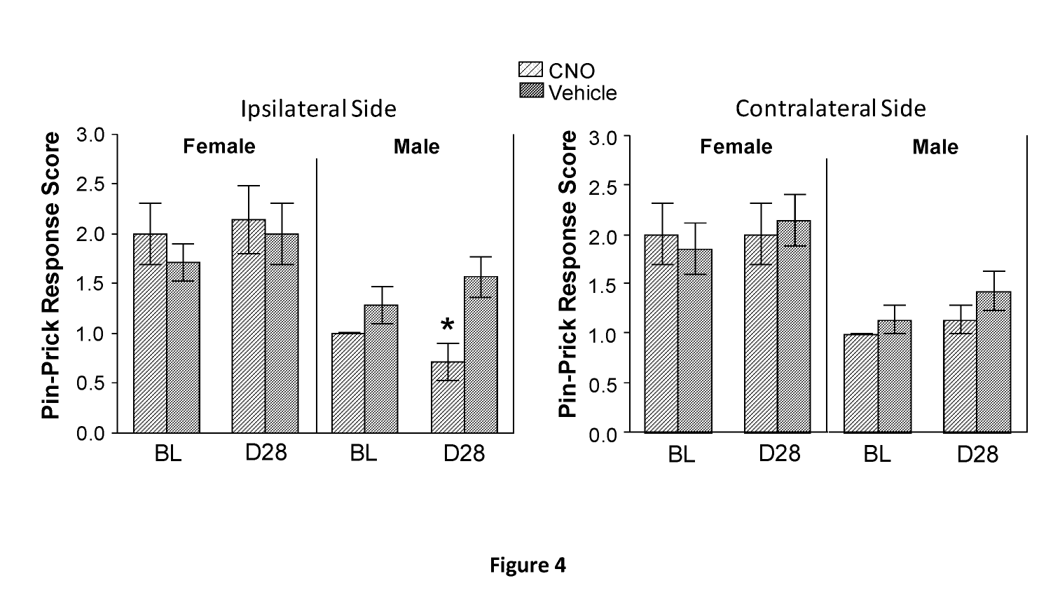

The effects of synapsin-DREADD activation with CNO on pin-prick response score were also studied (FIG. 4). The pin-prick assay is generally considered a nociceptive threshold test. All animals (males, n=7 per group; females, n=7 per group) had synapsin-DREADD (hSyn-hM4D) injected into the right trigeminal ganglion. Average pin-prick response scores (±SEM) are plotted following intraperitoneal (i.p.) administration of CNO or vehicle. Activation of hSyn-hM4D with CNO significantly reduced a response score to the pin-prick stimulation applied to the ipsilateral (right) vibrissal pad compared to the pin-prick response score in the ipsilateral side of the vehicle treated male rats (males: ipsilateral, ANOVA: treatment F=9.8, P=0.01). No significant effect of hSyn-hM4D activation with CNO on pin-prick response score was observed in the contralateral (left) vibrissal pad in male animals (males: contralateral, ANOVA: treatment F=1.3, P=0.3). In female animals, activation of hSyn-hM4D with CNO had no significant effect on response score to the pin-prick stimulation in either ipsilateral or contralateral sides compared to the ipsilateral or contralateral sides of vehicle treated females, respectively (females: ipsilateral, ANOVA: treatment F=0.1, P=0.8; contralateral, ANOVA: treatment F=0.1, P=0.7). This data also indicates that the synapsin promoter driven vector does not consistently and significantly affect normal (non-CCI) sensory detection thresholds.

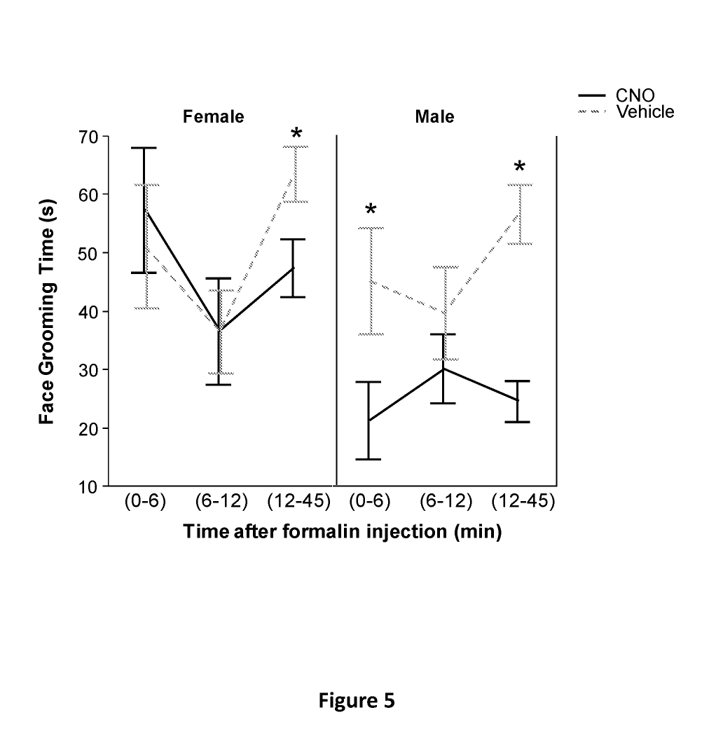

The effects of synapsin-DREADD activation with CNO on face grooming activity observed after subcutaneous (s.c.) injection of formalin into the right upper lip were also studied (FIG. 5). The mean number of seconds that rats (males, n=7 per group; females, n=7 per group) spent grooming (±SEM) is plotted for the 45 minutes post-formalin injection observation period. All animals had synapsin-DREADD (hSyn-hM4D) injected into the right trigeminal ganglion. CNO or vehicle was delivered intraperitoneally 30 minutes prior to the s.c. injection of 1.5% formalin (50 μl) into the right upper lip (ipsilateral side), just lateral to the nose. Following activation of hSyn-hM4D with CNO, total face grooming time was significantly reduced in the tonic phase (12-45 minutes post formalin injection) relative to the animals injected with the vehicle (in both male and female rats) (males: tonic phase, ANOVA: treatment F=27.1, P<0.0001); females: tonic phase, ANOVA: treatment F=5.5, P=0.02). Additionally, in males, activation of hSyn-hM4D with CNO resulted in a significant reduction of total face grooming time in the acute phase (0-6 minutes post formalin injection) relative to the animals injected with the vehicle (males: acute phase, ANOVA: treatment F=4.5, P=0.04; females: acute phase, ANOVA: treatment F=0.2, P=0.7). This indicates that the synapsin promoter driven vector significantly decreases pain responses induced by formalin.

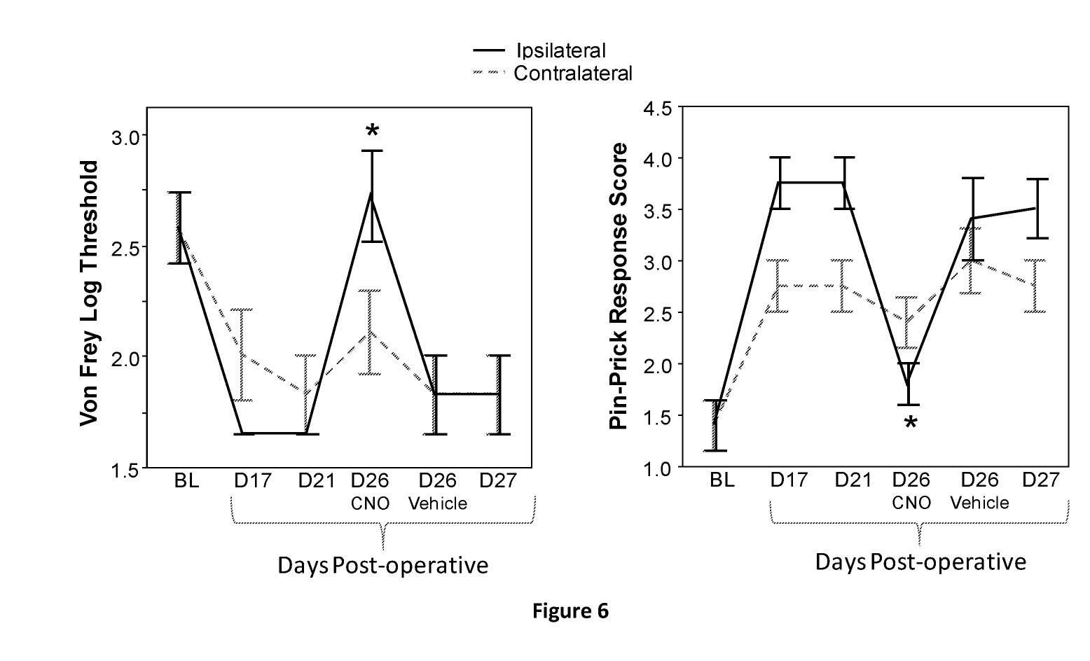

The effects of synapsin-DREADD activation with CNO on pain-like behavior following chronic constriction injury (CCI) of the infraorbital nerve (ION) were also studied (FIG. 6). The mean von Frey detection thresholds (±SEM) (left) and the mean response scores (±SEM) (right) to the pin-prick stimulation applied to the vibrissal pads were plotted. All animals (males, n=4 per group) were exposed to the infraorbital nerve chronic constriction injury (IoN-CCI) unilaterally. Following IoN-CCI, all animals had synapsin-DREADD (hSyn-hM4D) injected into the ipsilateral trigeminal ganglion. At post-operative day 26 (D26) CNO or vehicle was delivered intraperitoneally 30 minutes prior to behavioral testing. Activation of hSyn-hM4D with CNO significantly increased von Frey detection threshold compared to vehicle treated animals in the ipsilateral side (males: ipsilateral, ANOVA: treatment F=7.8, P=0.03). Activation of hSyn-hM4D with CNO significantly increased von Frey detection threshold in the ipsilateral side compared post-operative days 21 and 27 (males: ipsilateral D21, ANOVA: treatment F=20.4, P=0.004; ipsilateral D27, ANOVA: treatment F=7.8, P=0.03). Activation of hSyn-hM4D with CNO significantly decreased response score to the pin-prick stimulation applied to the ipsilateral vibrissal pad compared to vehicle treated animals (males: ipsilateral, ANOVA: treatment F=7.7, P=0.03). Activation of hSyn-hM4D with CNO significantly decreased response score to the pin-prick stimulation applied to the ipsilateral side compared to post-operative days 21 and 27 (males: ipsilateral D21, ANOVA: treatment F=32, P=0.001; ipsilateral D27, ANOVA: treatment F=21, P=0.004). No significant effects of hSyn-hM4D activation with CNO on von Frey detection threshold and pin-prick response score were observed in the contralateral side. These results show that the synapsin promoter driven vector decreases heightened painful behaviors that mimic trigeminal neuralgia elicited in CCI animals with hyperalgesia.

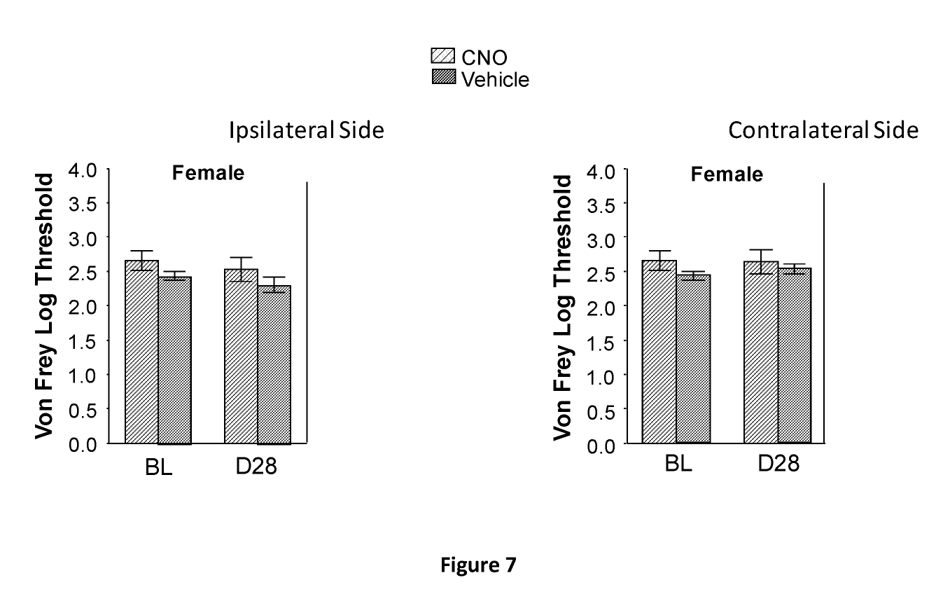

The effects of Tac1-DREADD activation with CNO on von Frey detection threshold were also studied (FIG. 7). All animals (female, n=7 per group) had Tachykinin 1-DREADD (hTac1-hM4D; Tac1 promoter driven DREADD) injected into the right trigeminal ganglion. Average von Frey detection thresholds (±SEM) are plotted following intraperitoneal administration of CNO or vehicle. In female rats, activation of hTac1-hM4D with CNO had no significant effect on von Frey detection thresholds in either ipsilateral or contralateral vibrissal pad compared to detection thresholds in the ipsilateral or contralateral sides of vehicle treated animals, respectively (females: ipsilateral, ANOVA: treatment F=1.4, P=0.3; contralateral, ANOVA: treatment F=0.4, P=0.6). This indicates that the Tac1 promoter driven vector has no significant effect on normal (non-CCI) sensory detection.

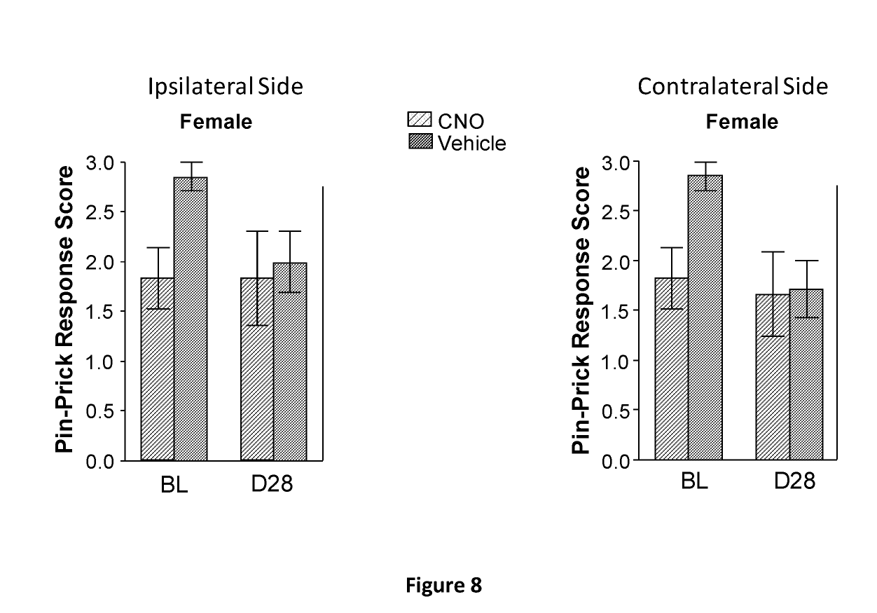

The effects of Tac1-DREADD activation with CNO on pin-prick response score were also studied (FIG. 8). All animals (female, n=7 per group) had Tachykinin 1-DREADD (hTac1-hM4D) injected into the trigeminal ganglion unilaterally. Average pin-prick response scores (±SEM) were plotted following intraperitoneal (i.p.) administration of CNO or vehicle. In female rats, activation of hTac1-hM4D with CNO had no significant effect on the response score to the pin-prick stimulation applied to either the ipsilateral or contralateral vibrissal pad compared to the response score in the ipsilateral or contralateral sides of vehicle treated animals, respectively (females: ipsilateral, ANOVA: treatment F=0.1, P=0.8; contralateral, ANOVA: treatment F=0.01, P=0.9). These results also indicate that the Tac1 promoter driven vector has no effect on normal (non-CCI) sensory detection.

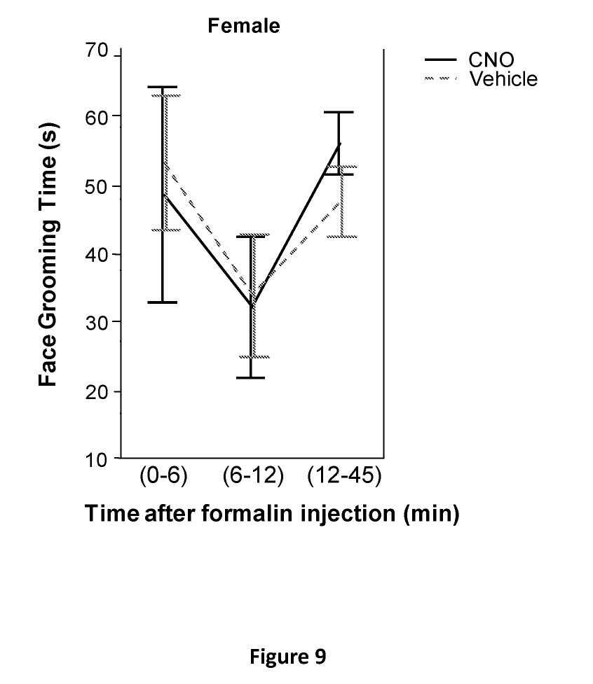

The effects of Tac1-DREADD activation with CNO on face grooming activity observed after s.c. injection of formalin into the right upper lip were also studied (FIG. 9). The mean number of seconds that rats (females, n=7 per group) spent grooming (±SEM) is plotted for the 45 minutes post-formalin injection observation period. All animals had Tac1-DREADD (hTac1-hM4D) injected into the right trigeminal ganglion. CNO or vehicle was delivered i.p. 30 minutes prior to the s.c. injection of 1.5% formalin (50 μl) into the right upper lip (ipsilateral side), just lateral to the nose. In females, activation of hTac1-hM4D with CNO had no significant effect on total face grooming time either the acute (0-6 minutes post formalin injection) or the tonic phase (12-45 minutes post formalin injection) relative to the animals injected with vehicle (females: acute phase, ANOVA: treatment F=0.07, P=0.8; tonic phase, ANOVA: treatment F=1.6, P=0.2). Surprisingly, the Tac1 promoter driven vector had no significant effect on formalin-induced pain response. This indicates that the formalin pain response is mediated principally by non-tachykinin circuits.

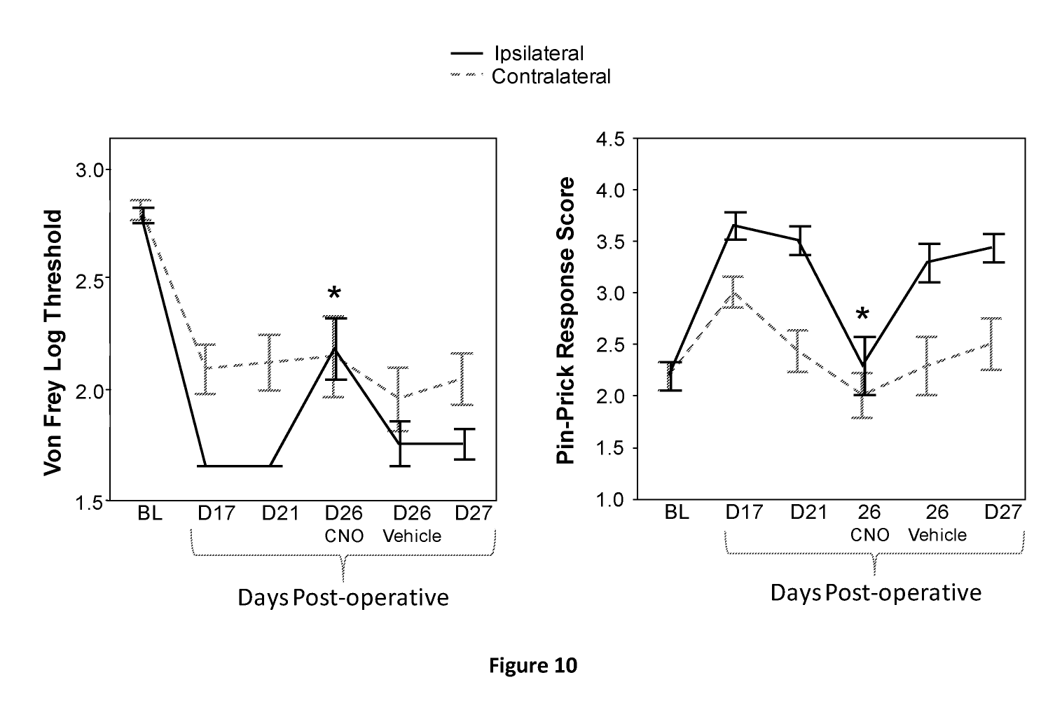

The effects of Tac1-DREADD activation with CNO on pain-like behavior following IoN-CCI were studied (FIG. 10). The mean von Frey detection thresholds (±SEM) (left) and the mean response scores (±SEM) (right) to the pin-prick stimulation applied to the vibrissal pads were plotted. All animals (males, n=7 per group) were exposed to the infraorbital nerve chronic constriction injury (IoN-CCI) unilaterally. Following IoN-CCI, all animals had tachykinin 1-DREADD (hTac1-hM4D) injected into the ipsilateral trigeminal ganglion. At post-operative day 26 (D26) CNO or vehicle was delivered intraperitoneally 30 minutes prior to behavioral testing. Activation of hTac1-hM4D with CNO significantly increased von Frey detection threshold compared to vehicle treated animals in the ipsilateral side (males: ipsilateral, ANOVA: treatment F=6.3, P=0.03). Activation of hTac1-hM4D with CNO significantly increased von Frey detection threshold in the ipsilateral side compared post-operative days 21 and 27 (males: ipsilateral D21, ANOVA: treatment F=31.4, P<0.0001; ipsilateral D27, ANOVA: treatment F=9.8, P=0.006). Activation of hTac1-hM4D with CNO also significantly decreased response score to the pin-prick stimulation applied to the ipsilateral vibrissaal pad compared to vehicle treated animals (males: ipsilateral, ANOVA: treatment F=8.6, P=0.01). Activation of hTac1-hM4D with CNO significantly decreased response score to the pin-prick stimulation applied to the ipsilateral side compared to post-operative days 21 and 27 (males: ipsilateral D21, ANOVA: treatment F=18.9, P=0.0003; ipsilateral D27, ANOVA: treatment F=16.9, P=0.0006). No significant effects of hSyn-hM4D activation with CNO on von Frey detection threshold and pin-prick response score were observed in the contralateral side. These data indicate that the Tac1 promoter driven vector decreases heightened painful behaviors that mimic trigeminal neuralgia elicited in CCI animals with hyperalgesia.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for inhibiting, treating, and/or preventing neuropathic pain in a subject, said method comprising:
a) administering to said subject a nucleic acid molecule encoding a Designer Receptor Exclusively Activated by Designer Drug (DREADD); and
b) administering an agonist of said DREADD to said subject after step a).

2. The method of claim 1, wherein said DREADD is Gi coupled.

3. The method of claim 1, wherein said DREADD is a Gi-coupled modified muscarinic receptor.

4. The method of claim 1, wherein said DREADD is hM4Di.

5. The method of claim 1, wherein said DREADD is a Gi-coupled modified kappa-opioid receptor.

6. The method of claim 1, wherein said nucleic acid molecule encoding DREADD is controlled by a neuron specific promoter.

7. The method of claim 6, wherein said neuron specific promoter is a tackykinin 1 promoter, synapsin promoter, or toll-like receptor 5 (TLRS) promoter.

8. (canceled)

9. (canceled)

10. The method of claim 6, wherein said nucleic acid molecule encoding DREADD is in a viral vector.

11. The method of claim 10, wherein said viral vector is an adeno-associated viral vector.

12. The method of claim 1, wherein said nucleic acid molecule encoding DREADD is administered by injection.

13. The method of claim 1, wherein step b) comprises administering said agonist orally, topically, or topically in a skin cream formulation.

14. (canceled)

15. (canceled)

16. The method of claim 3, wherein said agonist is clozapine N-oxide.

17. The method of claim 5, wherein said agonist is salvinorin B.

18. The method of claim 1, wherein said neuropathic pain is associated with a painful traumatic trigeminal neuropathy (PTTN) or neuropathic cancer pain.

19. (canceled)

20. A vector comprising a nucleic acid molecule encoding a Designer Receptor Exclusively Activated by Designer Drug (DREADD) controlled by a neuron specific promoter, wherein said DREADD is Gi coupled.

21. The vector of claim 20, wherein said DREADD is a Gi-coupled modified muscarinic receptor or a Gi-coupled modified kappa-opioid receptor.

22. The vector of claim 20, wherein said DREADD is hM4Di.

23. (canceled)

24. The vector of claim 20, wherein said neuron specific promoter is a tackykinin 1 promoter, synapsin promoter, or toll-like receptor 5 (TLR5) promoter.

25. (canceled)

26. (canceled)

27. The vector of claim 20, wherein said vector is a viral vector or an adeno-associated viral vector.

28. (canceled)

29. A composition comprising a vector of claim 20 and a pharmaceutically acceptable carrier.


SEQUENCE LISTING (1 sequence)

SEQ ID NO: 1 - DNA, 1228 bp, Artificial Sequence
  Tac1 promoter with upstream enhancer
gcggccgcacgcgtctgctgcagcaattcaaaggagaatcttgctgttcgggcagaagaaattcaatcaccttgtggagataatgaaaaagcttcatacttttaatcagatattgatcgattaccataatattctcccatagcaatagctgcaggcataagaaacggaaagaatggaagagatttttaggagaatacaaaaataaataagtatttgagacttagatactgcctttagtgacaagggtgaggatcctacacactatgttgctggtttcctagtcttcagcaagaaagtgtaggagagaagcaaaaaacgtcctgttcaacccctgctcctggatgtggcaaggaagaggagttacccggcttgaaacaaaagaaatcctaagtctgacacacaatgtcatgtttaaattcccctttctccaaaatgtaaaataaatctgcttccatcttctaaaatactatgggactaaacatccttttgttatgctaaggaaaagccagtattcgcgttgatttagaagagggatgttctggttatagaacgatgctgtgtctcagaaacacttaaatactattaagctagaaatagaagggaaaataatgcttccccgcatctcccctcaagtgtagtcctctttttttagcctgatttccgacgaaatgtctgaatgcctacagttatttggccatcctgaaaagtgcaacttatcctgacgtctcgagggacggaaaagttaccgaagtccaaggaatgagtcactttgctcaaatttgatgagtaatatcaggtgtcatgaaacccagtttcgaaggagaggggagggggcgtcagatctgcagacggaagcaggccgctccggattggatggcgagacctcgattttcctaaaattgcgtcatttagaacccaattgggtccagatgttatgggcatcgacgagttaccgtctcggaaactctcaatcacgcaagcgaaaggagaggaggcggctaattaaatattgagcagaaagtcgcgtggggagaatgtcacgtgggtctggaggctcaaggaggctgggataaataccgcaaggcactgagcaggcgaaagagcgcgctcggacctccttcccggcggcagctaccgagagtgcggagcgaccagcgtgcgctcggaggaaccagagaaactcagcaccccgcgggactgtccgtcgcagtaagtgggtaccggatcc